(19) United States
(12) Patent Application Publication
(10) Pub. No.: US 2021/0005313 A1
(43) Pub. Date: Jan. 7, 2021

(54) SAMPLE INSPECTION PLAN MANAGEMENT DEVICE, SAMPLE INSPECTION PLAN MANAGEMENT SYSTEM, SAMPLE INSPECTION PLAN MANAGEMENT METHOD, AND PROGRAM

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 17/031,525

(22) Filed: Sep. 24, 2020

Related U.S. Application Data
(63) Continuation of application No. PCT/JP2019/012126, filed on Mar. 22, 2019.

(30) Foreign Application Priority Data
Mar. 27, 2018 (JP) 2018-060602

(51) Int. Cl. G16H 40/20; G06Q 10/10; G16H 10/40; G06Q 10/08; G16H 15/00

(52) U.S. Cl. CPC G16H 40/20; G06Q 10/10; A61B 5/15; G06Q 10/0838; G16H 15/00; G16H 10/40

(57) ABSTRACT

Provided are a sample inspection plan management device, a sample inspection plan management system, a sample inspection plan management method, and a program capable of managing a sample inspection implemented by a doctor as a starting point. There is provided a sample inspection plan management device (12) that manages a sample inspection plan in a case where a sample inspection is implemented by using a sample inspection kit acquirable by a medical examinee outside a hospital. The device includes an inspection plan acquisition unit (30A) that acquires a sample inspection plan created by a doctor via a system of the hospital, a delivery information output unit (32A) that outputs delivery information including a delivery destination of the sample inspection kit based on the sample inspection plan, and an inspection result acquisition unit (30B) that acquires an inspection result of a sample collected by using the sample inspection kit delivered based on the delivery information via a system of an inspection institution that implements the sample inspection by retrieving the sample inspection kit.

### CROSS-REFERENCE TO RELATED APPLICATIONS

The present application is a Continuation of PCT International Application No. PCT/JP2019/012126 filed on Mar. 22, 2019 claiming priority under 35 U.S.C § 119(a) to Japanese Patent Application No. 2018-060602 filed on Mar. 27, 2018. Each of the above applications is hereby expressly incorporated by reference, in its entirety, into the present application.

### BACKGROUND OF THE INVENTION

### 1. Field of the Invention

The present invention relates to a sample inspection plan management device, a sample inspection plan management system, a sample inspection plan management method, and a program, and particularly to management of a sample inspection plan.

### 2. Description of the Related Art

The number of persons who desire to receive medical services but have difficulty in receiving the medical services has been increased due to an increase in the number of persons who desire the medical services due to aging, a decrease in the number of persons who work in a medical field due to a decrease in the number of children, a reduction in scale of medical institutions due to depopulation, and closure of the medical institutions due to depopulation.

In a case where persons who need to observe prognosis of disease regularly visit a hospital for inspection and listen to inspection results from a doctor, there are increasingly cases where these persons stay in the medical institutions for a long time, and thus, it is inefficient to provide the medical services.

The number of persons who have difficulty in visiting the hospitals and receive the medical services at home and the number of persons who have difficulty in visiting the hospitals and live in a remote area from the medical institutions have been increased. The persons who have difficulty in visiting the hospitals have difficulty in implementing necessary inspections.

JP2002-157332A describes a remote inspection method in which a patient mails a sample of the patient to an inspection facility, the inspection facility registers data of an inspection result, and the patient acquires the inspection result registered as the data via the Internet.

JP2005-224354A describes a health management system that supports health management by performing measurements at home by using a home biometric information measurement device such as a blood pressure monitor. In the health management system described in JP2005-224354A, in a case where a registrant inputs and transmits biometric information measured by using the home biometric information measurement device, the biometric information is transmitted to a medical specialist site. The medical specialist site feeds back a diagnosis result based on a diagnosis request from the registrant to the registrant. The diagnosis result is displayed by using a display unit of a registrant site and a display unit of a mobile phone.

### SUMMARY OF THE INVENTION

However, in the invention described in JP2002-157332A, a patient can implement a sample inspection without going to a medical institution such as a hospital and an inspection institution and a patient who has difficulty in visiting the hospital for inspection can implement the sample inspection. However, the sample is delivered and the inspection result is delivered between the patient and the inspection institution with no intervention of the doctor. That is, the invention described in JP2002-157332A is not a starting point of the inspection by the doctor.

In the invention described in JP2005-224354A, a delivery instruction of the home biometric information measurement device is issued from the medical specialist site, but the medical specialist site feeds back the diagnosis result based on the diagnosis request from the registrant. That is, in the invention described in JP2005-224354A, a medical specialist constituting the medical specialist site is also not the starting point of the inspection. That is, in both the invention described in JP2002-157332A and the invention described in JP2005-224354A, the sample inspection is implemented with the patient as the starting point based on the plan of the patient. By doing this, the doctor does not intervene to trigger the sample inspection, and it is difficult to implement the sample inspection according to the medical condition of the patient.

The present invention has been made in view of such circumstances, and an object thereof is to provide a sample inspection plan management device, a sample inspection plan management system, a sample inspection plan management method, and a program capable of managing a sample inspection implemented by a doctor as a starting point.

To achieve the above object, the following aspects of the invention are provided.

A sample inspection plan management device according to a first aspect is a sample inspection plan management device that manages a sample inspection plan in a case where a sample inspection is implemented by using a sample inspection kit acquirable by a medical examinee outside a hospital. The device comprises an inspection plan acquisition unit that acquires a sample inspection plan created by a doctor via a system of the hospital, a delivery information output unit that outputs delivery information including a delivery destination of the sample inspection kit based on the sample inspection plan, and an inspection result acquisition unit that acquires an inspection result of a sample collected by using the sample inspection kit delivered based on the delivery information via a system of an inspection institution that implements the sample inspection by retrieving the sample inspection kit.

According to the first aspect, the doctor creates the sample inspection plan using the sample inspection kit, and the sample inspection plan management device acquires the sample inspection plan created by the doctor. Accordingly, it is possible to manage the sample inspection plan using the sample inspection kit based on the sample inspection plan created by the doctor as the starting point.

The sample inspection kit capable of being acquired outside the hospital is used. Accordingly, the medical examinee who receives the inspection can implement the inspection without going to the hospital or the inspection institution, and activity efficiency of the medical examinee, the doctor, or the inspector can be improved.

The sample inspection plan created by the doctor can be acquired via the medical institution system. The medical institution system may be connected to communicate with a hospital terminal capable of being used by the doctor.

The sample inspection kit can apply the sample of the human. A sample capable of being collected by the medical examinee by using the sample inspection kit can be applied to the human sample. The sample inspection kit may apply a sample of animal other than human. A sample capable of being collected by an animal manager by using the sample inspection kit can be applied to the sample of animal other than human. Examples of the animal manager include owners and zoo caretakers.

An acquisition example of the sample inspection kit is the acquisition of the sample inspection kit delivered to the home at home. Other acquisition examples of the sample inspection kit include acquisition at a pharmacy, a store other than the pharmacy, a business office of a delivery company, and a post office.

The inspection institution retrieves the sample inspection kit from which the sample is collected, and implements the sample inspection. The inspection institution may include a sample inspection plan management unit.

A scheduling unit that manages the schedule of the sample inspection plan may be provided. The delivery information output unit may output the delivery information based on the schedule management of the sample inspection plan using the scheduling unit.

The sample inspection plan management device according to the first aspect is a sample inspection plan management device that comprises one or more processors and one or more memories. The one or more processors acquire a sample inspection plan created by a doctor via a system of a hospital, output delivery information including a delivery destination of the sample inspection kit based on the sample inspection plan, and acquire an inspection result of a sample collected by using the sample inspection kit delivered based on the delivery information via a system of an inspection institution that implements the sample inspection by retrieving the sample inspection kit. The memory can function as the sample inspection plan management device that stores data in various kinds of processing.

According to a second aspect, the sample inspection plan management device of the first aspect may further comprise an inspection result output unit that outputs the inspection result acquired by using the inspection result acquisition unit to the system of the hospital.

According to the second aspect, the system of the hospital can acquire the inspection result.

According to a third aspect, in the sample inspection plan management device of the second aspect, the inspection result output unit may output the inspection result acquired by using the inspection result acquisition unit to a medical examinee terminal device used by the medical examinee.

According to the third aspect, the medical examinee terminal device can acquire the inspection result.

According to a fourth aspect, in the sample inspection plan management device of any one aspect of the first aspect to the third aspect, the inspection plan acquisition unit may acquire the sample inspection plan including at least one of a kind of the sample, a frequency of the sample inspection, or the number of times of the sample inspection.

According to the fourth aspect, it is possible to manage the sample inspection plan including at least one of the kind of the sample, the frequency of the sample inspection, or the number of times of the sample inspection.

An inspection date can be specified based on the frequency of the sample inspection and the number of times of the sample inspection.

According to a fifth aspect, in the sample inspection plan management device of any one aspect of the first aspect to the fourth aspect, the inspection plan acquisition unit may acquire the sample inspection plan created by the doctor by using, as the sample, at least one of blood of human, feces of human, urine of human, blood of animal other than human, feces of animal other than human, or urine of animal other than human.

According to the fifth aspect, it is possible to manage the sample inspection plan that is created for the animal other than human by the doctor and uses the sample inspection kit.

The doctor includes a veterinarian.

According to a sixth aspect, in the sample inspection plan management device of any one aspect of the first aspect to the fifth aspect, the delivery information output unit may output the delivery information to a sample inspection kit providing institution that provides the sample inspection kit.

According to the sixth aspect, the sample inspection kit providing institution can transmit the sample inspection kit based on the sample inspection plan.

Examples of the sample inspection kit providing institution include an inspection institution, a manufacturer of the sample inspection kit, and a distributor of the sample inspection kit.

According to a seventh aspect, in the sample inspection plan management device of any one aspect of the first aspect to the fifth aspect, the delivery information output unit may output the delivery information to the inspection institution.

According to the seventh aspect, the sample inspection kit can be delivered from the inspection institution based on the sample inspection plan.

According to an eighth aspect, in the sample inspection plan management device of any one aspect of the first aspect to the seventh aspect, the delivery information output unit may output the delivery information having, as the delivery destination, any of home of the medical examinee, a pharmacy, a store other than the pharmacy, a business office of a delivery company, and a post office.

According to the eighth aspect, the medical examinee can acquire the sample inspection kit at the home of the medical examinee, the pharmacy, the store, the business office of the delivery company, or the post office.

Examples of a store other than the pharmacy include other stores that provide delivery services of the sample inspection kit such as convenience stores and supermarkets.

A sample inspection plan management system according to a ninth aspect is a sample inspection plan management system comprising a server device connected to communicate with a network. The server device is a sample inspection plan management device that manages a sample inspection plan in a case where a sample inspection is implemented by using a sample inspection kit acquirable by a medical examinee outside a hospital. The server device includes an inspection plan acquisition unit that acquires a sample inspection plan created by a doctor via a system of the hospital, a delivery information output unit that outputs delivery information including a delivery destination of the sample inspection kit based on the sample inspection plan, and an inspection result acquisition unit that acquires an inspection result of a sample collected by using the sample inspection kit delivered based on the delivery information via a system of an inspection institution that implements the sample inspection by retrieving the sample inspection kit.

According to the ninth aspect, the same effect as the effect in the first aspect can be obtained.

In the ninth aspect, matters similar to the matters specified in the second aspect to the eighth aspect can be appropriately combined. In this case, the components serving as the processing and functions specified in the sample inspection plan management device can be grasped as the components of the sample inspection plan management system serving as the corresponding processing and functions.

According to a tenth aspect, the sample inspection plan management system of the ninth aspect may further comprise a medical examinee terminal device that is connected to communicate with the network, and is used by the medical examinee. The medical examinee terminal device may include a display unit that displays at least one of a kind of the sample applied to the sample inspection, a frequency of the sample inspection, or the number of times of the sample inspection.

According to the tenth aspect, the medical examinee can grasp the contents of the sample inspection plan from the screen displayed by using the display unit of the medical examinee terminal device without going to the hospital and the inspection institution.

According to an eleventh aspect, in the sample inspection plan management system of the tenth aspect, the display unit may display a result of the sample inspection.

According to the eleventh aspect, the result of the sample inspection can be grasped from the screen displayed by using the display unit of the medical examinee terminal device.

According to a twelfth aspect, in the sample inspection plan management system of any one aspect of the ninth aspect to the eleventh aspect, a providing institution terminal device that is connected to communicate with the network, and is provided in a sample inspection kit providing institution that provides the sample inspection kit, may be further comprised. The delivery information output unit may output the delivery information to the providing institution terminal device.

According to the twelfth aspect, it is possible to output the delivery information of the sample inspection kit to the providing institution terminal based on the sample inspection plan. Accordingly, the sample inspection kit providing institution can deliver the sample inspection kit.

According to a thirteenth aspect, the sample inspection plan management system of any one aspect of the ninth aspect to the twelfth aspect may further comprise an inspection institution terminal device that is connected to communicate with the network, and is provided in the inspection institution. The delivery information output unit may output the delivery information to the inspection institution terminal device.

According to the thirteenth aspect, the delivery information of the sample inspection kit can be output to the inspection institution terminal based on the sample inspection plan. Accordingly, the inspection institution can deliver the sample inspection kit.

A sample inspection plan management method according to a fourteenth aspect is a sample inspection plan management method of managing a sample inspection plan in a case where a sample inspection is implemented by using a sample inspection kit acquirable by a medical examinee outside a hospital. The method comprises an inspection plan acquisition step of acquiring a sample inspection plan created by a doctor via a system of the hospital, a delivery information output step of outputting delivery information including a delivery destination of the sample inspection kit based on the sample inspection plan, and an inspection result acquisition step of acquiring an inspection result of a sample collected by using the sample inspection kit delivered based on the delivery information via a system of an inspection institution that implements the sample inspection by retrieving the sample inspection kit.

According to the fourteenth aspect, the same effect as the effect in the first aspect can be obtained.

In the fourteenth aspect, matters similar to the matters specified in the second aspect to the eighth aspect and the tenth aspect to the thirteenth aspect can be appropriately combined. In this case, the components serving as the processing and functions specified in the sample inspection plan management device and the sample inspection plan management system can be grasped as the components of the sample inspection plan management method serving as the corresponding processing and functions.

A program according to a fifteenth aspect is a program that manages a sample inspection plan in a case where a sample inspection is implemented by using a sample inspection kit acquirable by a medical examinee outside a hospital. The program causes a computer to realize an inspection plan acquisition function of acquiring a sample inspection plan created by a doctor via a system of the hospital, a delivery information output function of outputting delivery information including a delivery destination of the sample inspection kit based on the sample inspection plan, and an inspection result acquisition function of acquiring an inspection result of a sample collected by using the sample inspection kit delivered based on the delivery information via a system of an inspection institution that implements the sample inspection by retrieving the sample inspection kit.

According to the fifteenth aspect, the same effect as the effect in the first aspect can be obtained.

In the fifteenth aspect, matters similar to the matters specified in the second aspect to the eighth aspect and the tenth aspect to the thirteenth aspect can be appropriately combined. In this case, the components serving as the processing and functions specified in the sample inspection plan management device, the sample inspection plan management system, and the sample inspection plan management method can be grasped as the components of the program serving as the corresponding processing and functions.

According to the present invention, the doctor creates the sample inspection plan using the sample inspection kit, and the sample inspection plan management device acquires the sample inspection plan created by the doctor. Accordingly, it is possible to manage the sample inspection plan using the sample inspection kit based on the sample inspection plan created by the doctor as the starting point.

### BRIEF DESCRIPTION OF THE DRAWINGS

FIG. 1 is a block diagram showing a configuration example of a sample inspection plan management system.

FIG. 2 is a block diagram showing an example of a hardware configuration of a server device.

FIG. 3 is a functional block diagram of the server device.

FIG. 4 is a block diagram showing a configuration example of a hospital management system.

FIG. 5 is a flowchart showing a procedure flow of a sample inspection plan management method.

FIG. 6 is a flowchart showing a flow of information between components of the sample inspection plan management system and a flow of a sample inspection kit.

FIG. 7 is a block diagram showing a configuration example of a sample inspection plan management system to which a network system is applied.

FIG. 8 is an explanatory diagram showing an example of an inspection plan screen.

FIG. 9 is an explanatory diagram showing an example of an inspection result screen.

FIG. 10 is an explanatory diagram showing an example of a diagnosis result screen.

FIG. 11 is an explanatory diagram showing another example of the diagnosis result screen.

FIG. 12 is an explanatory diagram showing an example of a hospital visiting registration screen.

FIG. 13 is an explanatory diagram showing an example of a hospital visiting reservation screen.

FIG. 14 is an explanatory diagram showing an example of an inspection end screen.

### DESCRIPTION OF THE PREFERRED EMBODIMENTS

Hereinafter, preferred embodiments of the present invention will be described in detail with reference to the accompanying drawings. In the present specification, the same components are denoted by the same references, and thus, the redundant description is appropriately omitted.

[Sample Inspection Plan Management System]

[Overall Configuration]

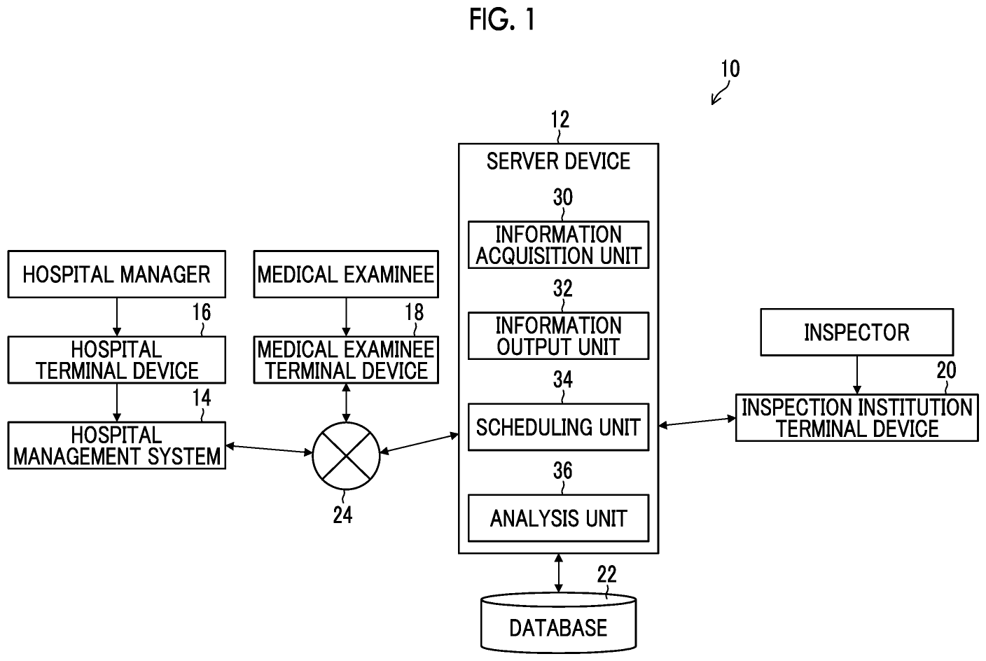

FIG. 1 is a block diagram showing a configuration example of a sample inspection plan management system. A sample inspection plan management system 10 shown in FIG. 1 manages a sample inspection plan created by a doctor according to an application from a medical examinee. A sample inspection plan is applied to a sample inspection kit capable of being acquired at a place other than a hospital such as home of the medical examinee and collecting a sample at a place other than the hospital such as the home of the medical examinee. The medical examinee includes a medical examinee who receives an inspection and an applicant for a sample inspection of a family of the medical examinee.

The sample inspection plan management system 10 shown in FIG. 1 comprises a server device 12, a hospital management system 14, a hospital terminal device 16, a medical examinee terminal device 18, an inspection institution terminal device 20, and a database 22. The server device 12, the hospital management system 14, and the medical examinee terminal device 18 are connected to communicate with each other via a network 24. The inspection institution terminal device 20 is an example of a system of an inspection institution.

The server device 12 is connected to communicate with the inspection institution terminal device 20 and the database 22. The server device 12, the inspection institution terminal device 20, and the database 22 can be connected in a wired or wireless manner.

The server device 12 and the database 22 may be arranged in the inspection institution, or may be arranged in a place different from the inspection institution. The server device 12 and the database 22 may be arranged in the same place, or may be arranged in different places. Different places may include different regions partitioned within the same building. The server device 12 corresponds to an example of a sample inspection plan management device.

The server device 12 comprises an information acquisition unit 30, an information output unit 32, a scheduling unit 34, and an analysis unit 36. The server device 12 comprises a storage unit (not shown). The storage unit (not shown) may be provided outside the server device 12.

The information acquisition unit 30 acquires information such as a sample inspection plan transmitted from the hospital management system 14 and information such as a sample inspection result transmitted from the inspection institution terminal device 20 via the network 24.

The information output unit 32 transmits information such as delivery information of the sample inspection kit to the inspection institution terminal device 20 via the network 24. The information output unit 32 transmits information such as the sample inspection result to the hospital management system 14 via the network 24. The information output unit 32 may transmit information such as the sample inspection result to the medical examinee terminal device 18 via the network 24.

In the present specification, data, information, a signal indicating data, and a signal indicating information can be interchanged with each other. A signal indicating data and a signal indicating information may be simply described as a signal.

Acquisition, reception, and input of the data, information, and signal can be interchanged with each other. Transmission and output of the data, information, and signal can be interchanged with each other.

The scheduling unit 34 monitors a schedule of the sample inspection plan acquired via the information acquisition unit 30.

The analysis unit 36 analyzes the result of the sample inspection transmitted from the inspection institution terminal device 20. The analysis result of the sample inspection result using the analysis unit 36 is stored in the database 22. The analysis unit 36 associates the medical examinee with the sample inspection result, and stores the analysis result of the sample inspection result in the database 22 for each medical examinee.

The hospital management system 14 is connected to communicate with the hospital terminal device 16. A connection form between the hospital management system 14 and the hospital terminal device 16 can be either a wired connection or a wireless connection.

The hospital management system 14 centrally manages the entire hospital. The hospital management system 14 is a collection of systems including a medical inspection system, an inspection system, and an accounting system. The hospital management system 14 may be called a hospital information system (HIS).

The hospital management system 14 is connected to communicate with one or more hospital terminal devices 16 arranged in the hospital. The hospital management system 14 manages information transmitted from the hospital terminal device 16 and information transmitted to the hospital terminal device 16.

The hospital terminal device 16 transmits information such as the sample inspection plan to the server device 12 via the hospital management system 14 and the network 24. The hospital terminal device 16 receives information such as the sample inspection result transmitted from the server device 12 via the hospital management system 14 and the network 24. The hospital management system 14 corresponds to an example of a hospital system.

Examples of the hospital terminal device 16 include a doctor terminal device used by a doctor, a nurse terminal device used by a nurse, and an accounting terminal device used by an accountant. A computer such as a personal computer or a tablet type terminal device can be applied to the hospital terminal device 16. As an example of the computer, there is a computer comprising a computer body, an operation member, and a display device. The same applies to the medical examinee terminal device 18 and the inspection institution terminal device 20 to be described below.

The medical examinee terminal device 18 is a terminal device used by the medical examinee. The medical examinee terminal device 18 receives information such as the sample inspection plan from the server device 12 via the network 24. The medical examinee terminal device 18 transmits information to the server device 12 via the network 24. A mobile terminal device such as a smartphone can be applied to the medical examinee terminal device 18.

The inspection institution terminal device 20 is one or more terminal devices arranged in the inspection institution. An example of the inspection institution is an inspection engineer terminal device used by an inspection engineer. The inspection institution terminal device 20 receives information such as the sample inspection plan from the server device 12 via the network 24. The inspection institution terminal device 20 transmits information such as the inspection result to the server device 12 via the network 24.

The database 22 stores various kinds of data managed by the server device 12. For example, the database 22 stores the sample inspection result for each medical examinee. The database 22 may store personal information for each medical examinee such as an inspection history for each medical examinee and a medical history for each medical examinee.

A public communication line network or a dedicated line network may be applied to the network 24. A standardized standard or a dedicated standard may be applied to the network 24.

[Hardware Configuration of Server Device]

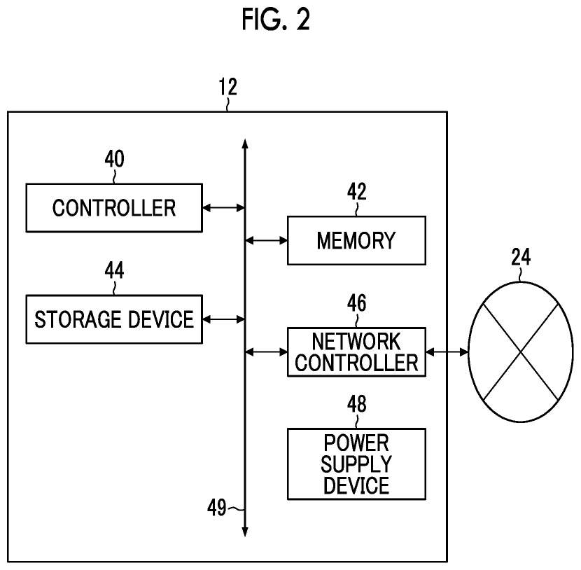

FIG. 2 is a block diagram showing an example of a hardware configuration of the server device. The server device 12 can realize the functions of the units of the server device 12 by executing a program by using hardware shown in FIG. 2.

The server device 12 comprises a controller 40, a memory 42, a storage device 44, a network controller 46, and a power supply device 48. The controller 40, the memory 42, the storage device 44, and the network controller 46 are connected via a bus 49 so as to perform data communication.

<Controller>

The controller 40 functions as an overall controller, various calculation units, and a storage controller of the server device 12. The controller 40 executes a program stored in a read only memory (ROM) included in the memory 42.

The controller 40 downloads a program from an external storage device via the network controller 46, and executes the downloaded program. The external storage device may be connected so as to be able to communicate with the server device 12 via a network 24.

The controller 40 uses a random access memory (RAM) included in the memory 42 as a calculation region, and executes various kinds of processing in cooperation with various programs. Accordingly, various functions of the server device 12 are realized.

The controller 40 controls the reading-out of data from the storage device 44 and the writing of data to the storage device 44. The controller 40 may acquire various kinds of data from an external storage device via the network controller 46. The controller 40 can execute various kinds of processing such as calculation by using the various kinds of acquired data.

The controller 40 may include one or two or more processors. Examples of the processor include a field programmable gate array (FPGA) and a programmable logic device (PLD). Circuit configurations of the FPGA and the PLD are changeable after being manufactured.

Another example of the processor is an application specific integrated circuit (ASIC). The ASIC has a circuit configuration specifically designed in order to execute specific processing.

The controller 40 can apply two or more processors of the same kind. For example, the controller 40 may use two or more FPGAs, or may use two PLDs. The controller 40 may apply two or more processors of different kinds. For example, the controller 40 may apply one or more FPGAs and one or more ASICs.

In a case where a plurality of controllers is provided, the plurality of controllers may be constituted by using one processor. As an example of a case where the plurality of controllers is constituted by one processor, there is a form in which one processor is constituted by using a combination of one or more central processing units (CPUs) and software and the processor functions as the plurality of controllers. A graphics processing unit (GPU) which is a processor specialized in image processing may be applied instead of the CPU or in combination with the CPU. The software mentioned herein is synonymous with the program. As a representative example in which the plurality of controllers is constituted by using one processor, there is a computer such as a client device and a server device.

As another example in which the plurality of controllers is constituted by one processor, there is a form in which a processor that realizes the functions of the entire system including the plurality of controllers by one IC chip is used. A representative example of the processor that realizes the functions of the entire system including the plurality of controllers, there is a system on chip (SoC). IC is an abbreviation for an Integrated Circuit.

In this manner, the controller 40 is constituted by using as one or more of various processors as a hardware structure.

<Memory>

The memory 42 comprises a ROM (not shown) and a RAM (not shown). The ROM stores various programs executed by the server device 12. The ROM stores parameters used for executing various programs, and files. The RAM functions as a temporary storage region of data and a work region of the controller 40.

<Storage Device>

The storage device 44 stores various kinds of data in a non-transitory manner. The storage device 44 may be externally attached to the outside of the server device 12. A large-capacity semiconductor memory device may be applied instead of or in combination with the storage device 44.

<Network Controller>

The network controller 46 controls data communication with external devices such as the hospital management system 14, the medical examinee terminal device 18, and the inspection institution terminal device 20 shown in FIG. 1. The control of the data communication may include management of traffic for the data communication.

<Power Supply Device>

A large-capacity power supply device such as an uninterruptible power supply (UPS) is applied as the power supply device 48. In a case where a commercial power supply is cut off due to a power failure, the power supply device 48 supplies a power to the server device 12. The hardware configuration of the server device 12 shown in FIG. 2 is an example, and addition, deletion, and change can be appropriately made.

[Function of Server Device]

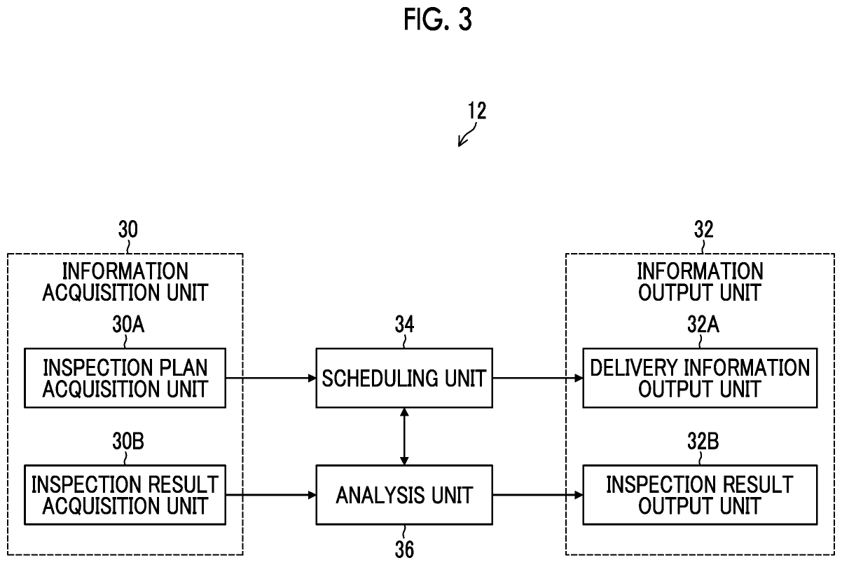

FIG. 3 is a functional block diagram of the server device. The server device 12 shown in FIG. 3 comprises the information acquisition unit 30, the information output unit 32, the scheduling unit 34, and the analysis unit 36. Hereinafter, the details of the units will be described.

<Information Acquisition Unit>

The information acquisition unit 30 comprises an inspection plan acquisition unit 30A and an inspection result acquisition unit 30B. The information acquisition unit 30 may comprise various information acquisition units that acquire various kinds of information. The illustration of the various information acquisition units is omitted.

Individual hardware or common hardware may be applied to the inspection plan acquisition unit 30A and the inspection result acquisition unit 30B. An example of a hardware configuration of the information acquisition unit 30, there is a configuration including a signal input element and a signal conversion element.

The inspection plan acquisition unit 30A acquires the sample inspection plan transmitted from the hospital management system 14. The inspection plan acquisition unit 30A stores the sample inspection plan in a prescribed storage unit (not shown). The inspection plan acquisition unit 30A transmits the sample inspection plan to the scheduling unit 34.

The inspection result acquisition unit 30B acquires the sample inspection result transmitted from the inspection institution terminal device 20. The sample inspection result acquired by using the inspection result acquisition unit 30B is transmitted to the analysis unit 36.

<Information Output Unit>

The information output unit 32 comprises a delivery information output unit 32A and an inspection result output unit 32B. The information output unit 32 may comprise various information output units that output various kinds of information. The illustration of the various information output units is omitted.

The delivery information output unit 32A transmits the delivery information of the sample inspection kit to the inspection institution terminal device 20 shown in FIG. 1. The sample inspection kit delivery information includes a kind of the sample inspection kit, a delivery destination of the sample inspection kit, and a delivery date of the sample inspection kit. The delivery date of the sample inspection kit may be a date at which the sample inspection kit is delivered from the delivery source or a date at which the sample inspection kit arrives at the delivery destination.

The inspection result output unit 32B transmits the inspection result to the hospital management system 14 shown in FIG. 1. The inspection result output unit 32B may transmit the inspection result to the medical examinee terminal device 18.

<Scheduling Unit>

The scheduling unit 34 registers the sample inspection plan acquired by using the inspection plan acquisition unit 30A, and manages the schedule of the sample inspection plan. The schedule of the sample inspection plan includes the number of times of the sample inspection and a frequency of the sample inspection. The scheduling unit 34 decides the delivery date of the sample inspection kit based on the number of times of the sample inspection and the frequency of the sample inspection.

The scheduling unit 34 manages an inspection result acquisition date at which the inspection result is acquired from the inspection institution terminal device 20 and an inspection result transmission date at which the inspection result is transmitted to the hospital management system 14. In a case where the inspection result cannot be acquired from the inspection institution terminal device 20 even after the inspection result acquisition date, the scheduling unit 34 may transmit a warning to the inspection institution terminal device 20.

<Analysis Unit>

The analysis unit 36 analyzes the inspection result transmitted from the inspection institution terminal device 20. The analysis unit 36 determines whether or not the sample inspection is implemented based on the sample inspection plan for an inspection condition other than the schedule. The analysis unit 36 stores the inspection result in the database 22.

In a case where the sample inspection is not implemented based on the sample inspection plan for the inspection condition other than the schedule, the analysis unit 36 may transmit a warning to the inspection institution terminal device 20. The scheduling unit 34 and the analysis unit 36 shown in FIG. 3 may be integrated.

[Configuration Example of Hospital Management System]

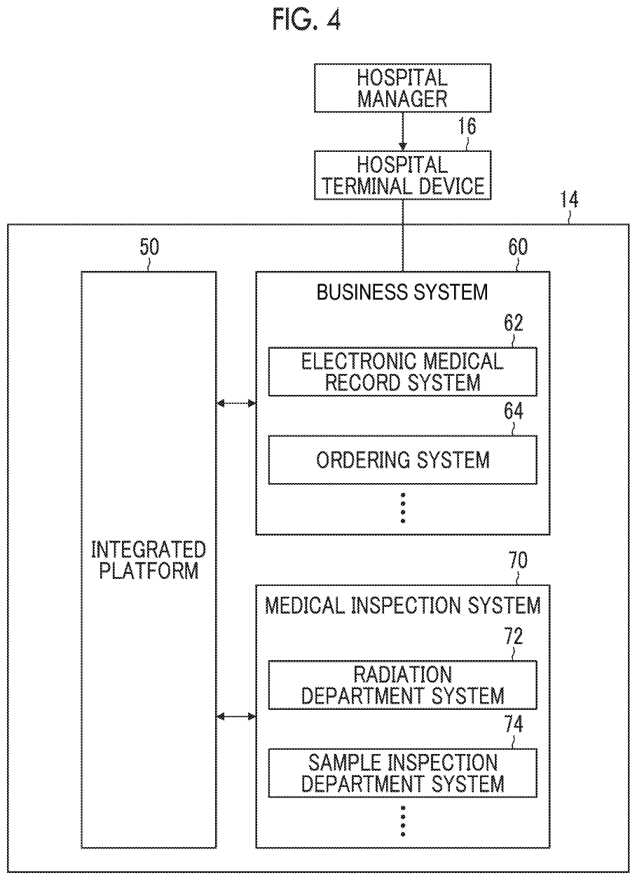

FIG. 4 is a block diagram showing a configuration example of the hospital management system. The same configuration as the configuration of the server device 12 shown in FIG. 2 can be applied to the hardware configuration of the hospital management system 14 shown in FIG. 4. Here, the description of the hardware configuration of the hospital management system 14 is omitted.

The hospital management system 14 can realize the functions of the units by executing the program by using hardware such as a processor. The hospital management system 14 comprises an integrated platform 50, a business system 60, and a medical inspection system 70.

The integrated platform 50 centrally controls the business system 60 and the medical inspection system 70. The integrated platform 50 functions as an integrated interface that integrates the business system 60 and the medical inspection system 70.

The business system 60 comprises an electronic medical record system 62 and an ordering system 64. The business system 60 may comprise a nursing support system (not shown). The electronic medical record system 62 manages an electronic medical record. The ordering system 64 manages inspections performed by doctors and orders such as prescriptions.

The medical inspection system 70 comprises a radiation department system 72, and a sample inspection department system 74. The medical inspection system 70 may comprise systems of departments related to medical inspections or inspections such as an endoscopic department system and a physiological inspection department system (not shown).

The sample inspection plan transmitted from the hospital terminal device 16 to the hospital management system 14 can be output from the hospital management system 14 via the ordering system 64.

[Procedure for Sample Inspection Plan Management Method]

[Procedure of Sample Inspection Plan Management Method Executed by Server Device]

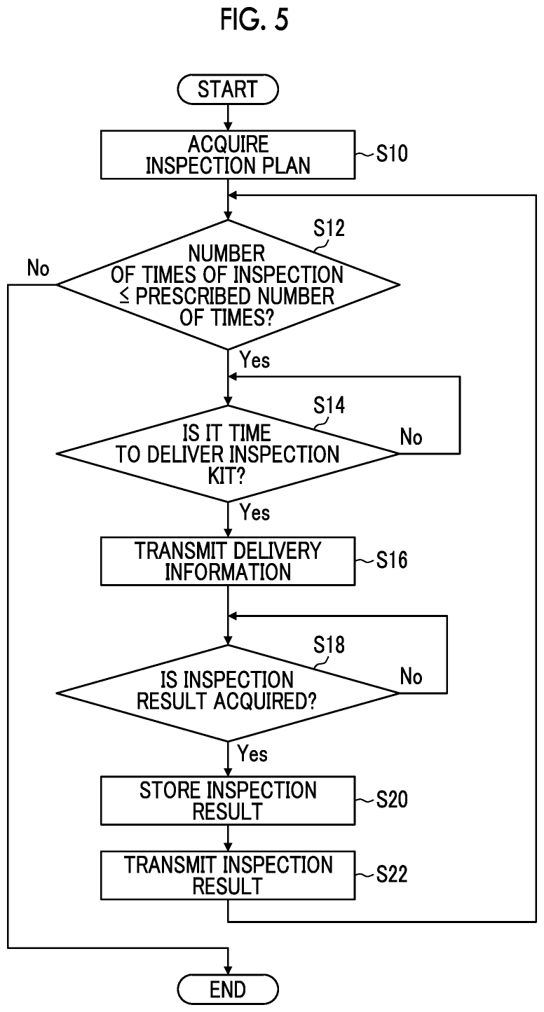

FIG. 5 is a flowchart showing a procedure flow of a sample inspection plan management method. A procedure of the sample inspection plan management method shown in FIG. 5 can be realized by executing a program in the server device 12 shown in FIG. 1.

In inspection plan acquisition step S10, the inspection plan acquisition unit 30A shown in FIG. 4 acquires the sample inspection plan transmitted from the hospital management system 14 shown in FIG. 1. After inspection plan acquisition step S10, the processing proceeds to inspection number-of-times determination step S12. A registration step of registering the acquired inspection result as a management target may be executed after inspection plan acquisition step S10. After the registration step, inspection number-of-times determination step S12 may be performed.

In inspection number-of-times determination step S12, the scheduling unit 34 determines whether or not the number of times of the inspection is equal to or less than a prescribed number of times determined in the sample inspection plan. In inspection number-of-times determination step S12, the sample inspection is performed by the prescribed number of times, and in a case where the number of times of the inspection exceeds the prescribed number of times, the determination result is No. In a case where the determination result is No, the server device 12 ends the sample inspection plan management method.

Meanwhile, in a case where the number of times of the inspection is equal to or less than the prescribed number of times in inspection number-of-times determination step S12, the determination result is Yes. In a case where the determination result is Yes, the processing proceeds to inspection kit delivery timing determination step S14. In inspection kit delivery timing determination step S14, the scheduling unit 34 determines whether or not it is time to deliver the inspection kit. In a case where the scheduling unit 34 determines that it is not time to deliver the inspection kit in inspection kit delivery timing determination step S14, the determination result is No. In a case where the determination result is No, inspection kit delivery timing determination step S14 is executed until the determination result is Yes in inspection kit delivery timing determination step S14.

Meanwhile, the scheduling unit 34 determines that it is time to deliver the inspection kit in inspection kit delivery timing determination step S14, the determination result is Yes. In a case where the determination result is Yes, the processing proceeds to delivery information transmission step S16.

In delivery information transmission step S16, the scheduling unit 34 transmits the delivery information of the sample inspection kit to the inspection institution terminal device 20 via the delivery information output unit 32A. After delivery information transmission step S16, the processing proceeds to inspection result acquisition step S18. Delivery information transmission step S16 corresponds to an example of a delivery information output step.

In inspection result acquisition step S18, the inspection result acquisition unit 30B determines whether or not the inspection result is acquired from the inspection institution terminal device 20. In a case where the scheduling unit 34 does not acquire the inspection result in inspection result acquisition step S18, the determination result is No.

In a case where the determination result is No, inspection result acquisition step S18 is executed until the inspection result acquisition unit 30B acquires the inspection result from the inspection institution terminal device 20 and the determination result is Yes in inspection result acquisition step S18. Meanwhile, in a case where the inspection result acquisition unit 30B acquires the inspection result in inspection result acquisition step S18, the determination result is Yes. In a case where the determination result is Yes, the processing proceeds to inspection result storing step S20.

In inspection result storing step S20, the analysis unit 36 stores the inspection result transmitted from the inspection institution terminal device 20 in the database 22. After inspection result storing step S20, the processing proceeds to inspection result transmission step S22.

In inspection result transmission step S22, the inspection result output unit 32B transmits the inspection result to the hospital management system 14. In inspection result transmission step S22, the inspection result output unit 32B may transmit the inspection result to the medical examinee terminal device 18.

After inspection result transmission step S22, the processing proceeds to inspection number-of-times determination step S12. Hereinafter, the steps from inspection number-of-times determination step S12 to inspection result transmission step S22 are repeatedly executed until the prescribed number of times of the sample inspections are ended and the determination result is No in inspection number-of-times determination step S12.

[Flow of Information and Flow of Sample Inspection Kit in Sample Inspection Management System]

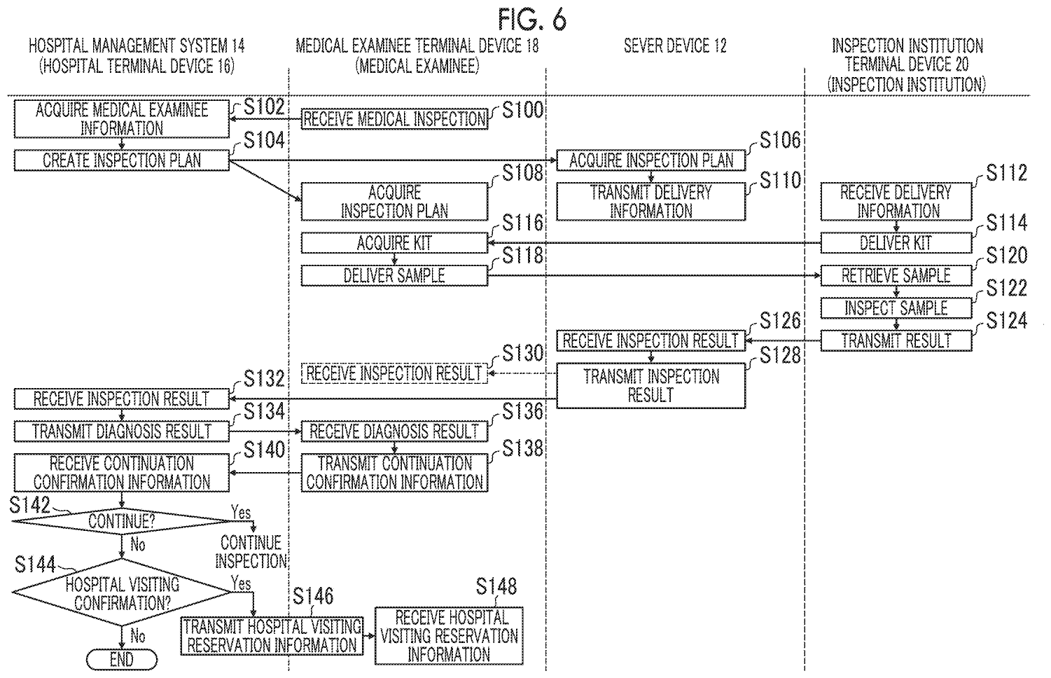

FIG. 6 is a flowchart showing a flow of information between the components of the sample inspection plan management system and a flow of the sample inspection kit. First, in medical inspection step S100, the medical examinee receives a medical inspection of the doctor at the hospital. The medical examinee information input by the doctor to the electronic medical record during the medical inspection is registered in a database (not shown) of the hospital management system 14 in medical examinee information acquisition step S102.

The medical examinee information input to the electronic medical record is stored in the database (not shown) for each medical examinee. In a case where there is the medical examinee information in the database, the medical examinee information newly input to the electronic medical record is added.

In inspection plan creation step S104, the doctor creates the sample inspection plan of the medical examinee. In a case where it is difficult for the medical examinee to regularly receive the sample inspection, the doctor creates the sample inspection plan using the sample inspection kit. A case where it is difficult to receive regular medical inspections includes a case where the medical examinee lives in a remote area from the hospital, a case where the medical examinee is elderly, and a case where the medical examinee has a disability.

In inspection plan acquisition step S106, the server device 12 acquires the sample inspection plan transmitted from the hospital management system 14. In inspection plan acquisition step S108, the medical examinee terminal device 18 acquires the sample inspection plan transmitted from the hospital management system 14. Inspection plan acquisition step S106 shown in FIG. 6 corresponds to inspection plan acquisition step S10 shown in FIG. 5.

In delivery information transmission step S110, the server device 12 transmits the delivery information of the sample inspection kit to the inspection institution terminal device 20. Delivery information transmission step S110 shown in FIG. 6 corresponds to delivery information transmission step S16 shown in FIG. 5.

In delivery information reception step S112, the inspection institution terminal device 20 receives the delivery information transmitted from the server device 12. In kit delivery step S114, the inspection institution terminal device 20 delivers the sample inspection kit of the kind designated for the delivery information to the delivery destination designated for the delivery information on the delivery date designated for the delivery information. Kit delivery step S114 may be implemented by an external institution of the inspection institution such as a case of being implemented by the delivery institution of the sample inspection kit requested by the inspection institution and a manufacturing institution of the sample inspection kit requested by the inspection institution. The inspection institution corresponds to an example of a sample inspection kit providing institution. The external institution of the inspection institution such as the manufacturing institution of the sample inspection kit corresponds to another example of the sample inspection kit providing institution. The inspection institution terminal device 20 corresponds to an example of a providing institution terminal device.

In kit acquisition step S116, the medical examinee acquires the sample inspection kit delivered from the inspection institution. The acquisition of the sample inspection kit is synonymous with the reception of the sample inspection kit. The sample inspection kit can be obtained by using home of the applicant, a pharmacy, a store other than the pharmacy, a business office of a delivery company, or a post office.

The medical examinee who acquires the sample inspection kit collects the sample by using the sample inspection kit. The collection of the sample is implemented based on an instruction manual attached to the sample inspection kit. Here, the description of the collection of the sample is omitted.

In sample delivery step S118, the medical examinee delivers the collected sample to the inspection institution. The sample is delivered based on the instruction manual attached to the sample inspection kit. Here, the description of the delivery of the sample is omitted.

In sample retrieving step S120, the inspection institution retrieves the sample delivered from the medical examinee. The retrieving of the sample is synonymous with the acquisition of the sample. In the present specification, the retrieving of the sample and the acquisition of the sample can be interchanged with each other.

In sample inspection step S122, the inspection institution implements the sample inspection, and creates the sample inspection result. In the sample inspection, sample inspection item values may be derived for all sample inspection items capable of being derived from the acquired sample, or the sample inspection item values may be derived for some of the sample inspection items capable of being derived from the acquired sample.

In result transmission step S124, the inspection institution terminal device 20 transmits the sample inspection result to the server device 12.

In inspection result reception step S126, the server device 12 receives the sample inspection result transmitted from the inspection institution terminal device 20. Inspection result reception step S126 corresponds to an inspection result acquisition step S18 shown in FIG. 5.

In inspection result transmission step S128, the server device 12 transmits the sample inspection result to the hospital management system 14. In inspection result transmission step S128, the server device 12 may transmit the sample inspection result to the medical examinee terminal device 18. Inspection result transmission step S128 corresponds to an inspection result transmission step S22 shown in FIG. 5.

In a case where the sample inspection result is transmitted from the server device 12 to the medical examinee terminal device 18, inspection result reception step S130 is executed. In inspection result reception step S130, the medical examinee terminal device 18 receives the sample inspection result transmitted from the server device 12. The medical examinee terminal device 18 can display the sample inspection result by using a display unit.

In inspection result reception step S132, the hospital management system 14 receives the sample inspection result transmitted from the server device 12. The doctor creates a diagnosis result based on the sample inspection result. The diagnosis result is registered in the hospital management system 14 from the hospital terminal device 16 used by the doctor. The diagnosis result may include the sample inspection result.

In diagnostic result transmission step S134, the hospital management system 14 transmits the diagnosis result to the medical examinee terminal device 18. In diagnostic result reception step S136, the medical examinee terminal device 18 receives the diagnosis result transmitted from the hospital management system 14. The medical examinee terminal device 18 can display the diagnosis result by using the display unit.

In continuation confirmation information transmission step S138, the medical examinee terminal device 18 transmits continuation confirmation information indicating whether or not to continue the sample inspection to the hospital management system 14. That is, the medical examinee who sees the diagnosis result transmits the continuation confirmation information to the hospital management system 14.

In continuation confirmation information reception step S140, the hospital management system 14 receives the continuation confirmation information transmitted from the medical examinee terminal device 18. In continuation confirmation step S142, the hospital management system 14 determines whether to continue the sample inspection or to stop the sample inspection.

For example, in a case where the medical examinee who sees the diagnosis result registers a visit to the hospital, the medical examinee can transmit the continuation confirmation information including hospital visiting registration information to the hospital management system 14 by using the medical examinee terminal device 18.

Meanwhile, in a case where the sample inspection is continued, the medical examinee can transmit the continuation confirmation information indicating the continuation of the sample inspection to the hospital management system 14 by using the medical examinee terminal device 18.

The determination result is No in a case where the sample inspection is stopped in continuation confirmation step S142, and in a case where the determination result is No, the processing proceeds to hospital visiting confirmation step S144. Meanwhile, in a case where the continuation confirmation information indicates the continuation of the sample inspection in continuation confirmation step S142, the determination result is Yes, and in a case where the determination result is Yes, the sample inspection is continued based on the sample inspection plan.

In hospital visiting confirmation step S144, the hospital management system 14 determines whether or not the hospital visiting registration information transmitted from the medical examinee is received. In a case where the hospital visiting registration information is not received in hospital visiting confirmation step S144, the determination result is No. In a case where the determination result is No, the hospital management system 14 waits for the reception of the hospital visiting registration information for a certain period. In a case where the hospital visiting registration information is not received for a certain period, the hospital management system 14 transmits management end information indicating that the sample inspection plan management method is ended to the server device 12. The server device 12 that receives the management end information ends the sample inspection plan management method.

Meanwhile, in a case where the hospital visiting registration information transmitted from the medical examinee is received in hospital visiting confirmation step S144, the determination result is Yes. In a case where the determination result is Yes, the hospital management system 14 confirms the hospital visiting reservation of the medical examinee based on the hospital visiting registration information transmitted from the medical examinee terminal device 18. In hospital visiting reservation information transmission step S146, the hospital management system 14 transmits the hospital visiting reservation information indicating that the hospital visiting reservation is confirmed to the medical examinee terminal device 18.

In hospital visiting reservation information reception step S148, in a case where the medical examinee terminal device 18 receives the hospital visiting reservation information transmitted from the hospital management system 14, the medical examinee terminal device 18 displays the hospital visiting reservation information by using the display unit.

The server device 12 repeatedly executes the steps from kit delivery step S114 to hospital visiting reservation information reception step S148 until it is determined that the number of times of the sample inspection reaches the prescribed number of times.

In a case where the doctor creates change information for changing the sample inspection plan and the change information is registered in the hospital management system 14, the change information indicating the change content of the sample inspection plan may be transmitted from the hospital management system 14 to the server device 12.

In a case where the server device 12 receives the change information transmitted from the hospital management system 14, the server device 12 can change the sample inspection plan based on the change information. The server device 12 can transmit the changed sample inspection plan to the medical examinee terminal device 18.

[Operations and Effects of Sample Inspection Plan Management Device, System, and Method]

According to the sample inspection plan management device, the sample inspection plan management system, and the sample inspection plan management method having the aforementioned configuration, the following operations and effects can be obtained.

[1]

The doctor creates the sample inspection plan using the sample inspection kit, and the server device 12 acquires the sample inspection plan created by the doctor. Accordingly, it is possible to manage the sample inspection plan based on the sample inspection plan created by the doctor.

[2]

The medical examinee collects the sample by using the sample inspection kit delivered from the inspection institution. The medical examinee delivers the collected sample to the inspection institution. Accordingly, the medical examinee can implement the sample inspection without going to the hospital and the inspection institution.

[3]

The sample inspection plan prescribes at least one of a kind of the sample, a frequency of the sample inspection, or the number of times of the sample inspection. Accordingly, the sample inspection plan is managed based on at least one of the kind of the sample, the frequency of the sample inspection, or the number of times of the sample inspection.

[4]

The delivery information of the sample inspection kit is delivered to the inspection institution. Accordingly, the inspection institution can deliver the sample inspection kit based on the sample inspection plan. Instead of the inspection institution, the sample inspection kit providing institution may receive the delivery information of the sample inspection kit, and may deliver the sample inspection kit.

[5]

The server device 12 transmits the inspection result transmitted from the inspection institution to the hospital management system 14. Accordingly, the doctor can create the diagnosis result based on the inspection result.

[6]

The diagnosis result is transmitted to the medical examinee terminal device 18. Accordingly, the medical examinee can acquire the diagnosis result created by the doctor based on the inspection result.

[Application Example to Network System]

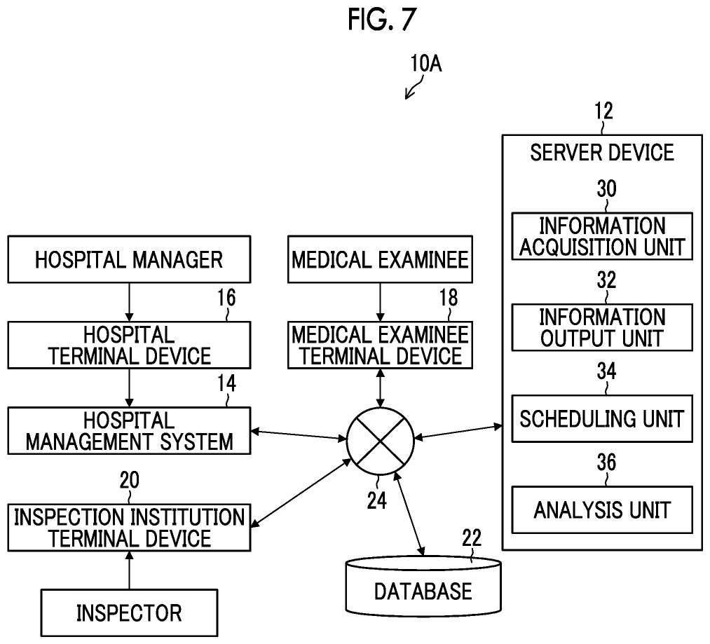

FIG. 7 is a block diagram showing a configuration example of a sample inspection plan management system to which the network system is applied. A sample inspection plan management system 10A shown in FIG. 7 is different from the sample inspection plan management system 10 shown in FIG. 1 in that the server device 12, the inspection institution terminal device 20, and the database 22 are connected to communicate with each other via the network 24.

The components of the sample inspection plan management system 10A are the same as the components of the sample inspection plan management system 10 shown in FIG. 1. Here, the description of each component of the sample inspection plan management system 10A is omitted.

That is, in the sample inspection plan management system 10A, the server device 12, the hospital management system 14, the medical examinee terminal device 18, and the inspection institution terminal device 20 which are the components of the sample inspection plan management system 10A are connected to communicate with each other via the network 24. The server device 12 and the database 22 are connected to communicate with each other via the network 24.

Accordingly, the server device 12 can be arranged in a place different from the inspection institution. The database 22 can be arranged in a place different from the server device 12. For example, the inspection institution may be arranged in Japan, and the server device 12 may be arranged in a foreign country. The foreign country indicates countries or regions other than Japan.

[Screen Display of Medical Examinee Terminal Device]

Next, the screen display displayed on the display unit included in the medical examinee terminal device 18 will be described. In the screen display to be described below, a display content and a display format such as a character size may be automatically changed according to a kind of the display unit and performance of the display unit such as a screen size of the display unit.

[Configuration Example of Inspection Plan Screen]

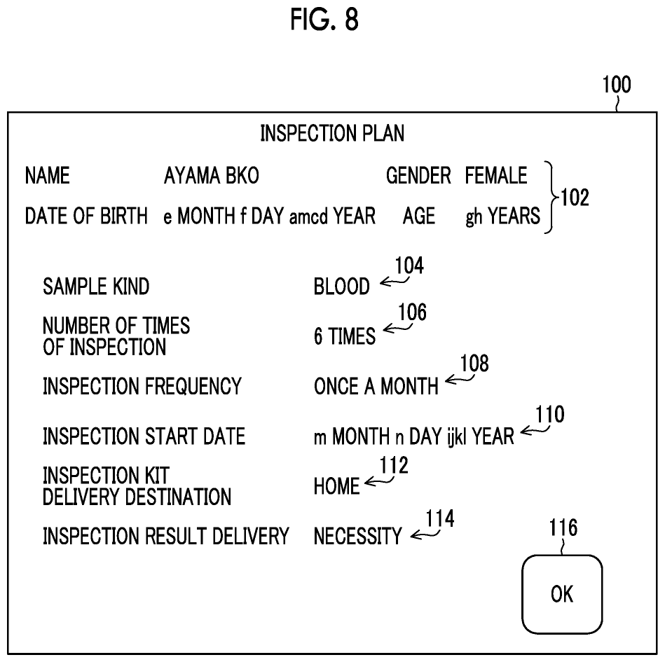

FIG. 8 is an explanatory diagram showing an example of an inspection plan screen. An inspection plan screen 100 shown in FIG. 8 is generated based on the sample inspection plan transmitted from the server device 12. The inspection plan screen 100 can be displayed by using the display unit included in the medical examinee terminal device 18 shown in FIG. 1. The inspection plan screen 100 is displayed on the display unit included in the medical examinee terminal device 18 after inspection plan acquisition step S108 shown in FIG. 6.

Applicant information 102, sample kind information 104, inspection number-of-times information 106, inspection frequency information 108, inspection start date information 110, inspection kit delivery destination information 112, inspection result delivery necessity information 114, and OK button 116 are displayed on the inspection plan screen 100.

The applicant information 102 shown in FIG. 8 includes a name of the applicant, a gender of the applicant, a date of birth of the applicant, and an age of the applicant. The applicant information 102 may include other information capable of being used to specify the applicant.

The sample kind information 104 displays the kind of the sample. Examples of the kind of the sample include blood, urine, feces, saliva, mucous membrane, and hair. An animal sample other than human may be applied to the sample.

The inspection number-of-times information 106, the inspection frequency information 108, the inspection start date information 110, the inspection kit delivery destination information 112, and the inspection result delivery necessity information 114 are displayed based on the inspection plan. The inspection plan screen 100 may display inspection conditions other than the inspection number-of-times information 106 shown in FIG. 8.

The OK button 116 is a button operated in a case where the medical examinee closes the inspection plan screen 100. In a case where the OK button 116 is clicked, the inspection plan screen 100 is closed.

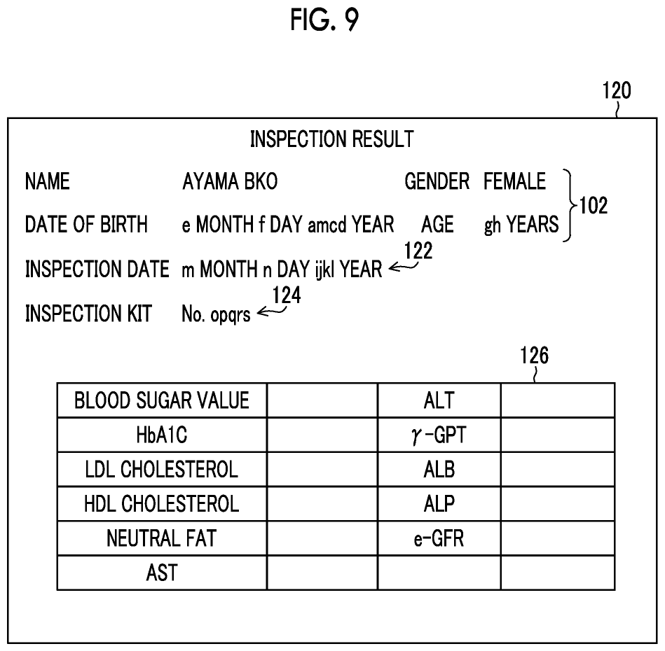

Alphabets shown in FIG. 8 indicate any characters, numbers, and alphabets. Information based on the sample inspection plan is displayed as each information shown in FIG. 8. The same applies to FIGS. 9 to 14.

[Configuration Example of Inspection Result Screen]

FIG. 9 is an explanatory diagram showing an example of an inspection result screen. An inspection result screen 120 shown in FIG. 9 is generated based on the sample inspection result transmitted from the inspection institution terminal device 20. The inspection result screen 120 can be displayed by using the display unit included in the medical examinee terminal device 18 after inspection result reception step S130 shown in FIG. 6.

The inspection result screen 120 shown in FIG. 9 includes the applicant information 102, inspection date information 122, inspection kit information 124, and an inspection result list 126.

The inspection date information 122 may be a sample collection date at which the medical examinee collected the sample, or may be a sample analysis date at which the inspection institution analyzes the sample. At least one of the sample collection date or the sample analysis date may be displayed on the inspection result screen 120.

The inspection kit information 124 is information that specifies the sample kit delivered to the medical examinee. The inspection kit information 124 shown in FIG. 9 is a serial number of the sample inspection kit. A management number of the sample inspection kit and a production lot number of the sample inspection kit may be applied to the inspection kit information 124.

Item values for each sample inspection item are displayed in the inspection result list 126. The inspection result list 126 shown in FIG. 9 is in a list form. The format of the inspection result list 126 is not limited. For example, a graph format may be applied. All the sample inspection items or a part of the sample inspection items may be displayed in the inspection result list 126.

Blood sugar, HbA1c, HDL cholesterol, LDL cholesterol, neutral fat, AST, ALT, γ-GPT, ALP, ALB, and e-GFR are illustrated as the blood inspection item in the inspection result list 126 of FIG. 9.

HbA1c indicates glycohemoglobin. HDL is an abbreviation for high density lipoprotein. LDL is an abbreviation for low density lipoprotein.

AST indicates aspartate aminotransferase. ALT indicates alanine aminotransferase. γ-GPT indicates gamma glutamyl transpeptidase. ALB indicates albumin. ALP indicates alkaline phosphatase. e-GFR indicates an estimated glomerular filtration rate.

Examples of the inspection item in a case where the sample is urine include urinary protein, urine occult blood, and urobilinogen. Examples of the inspection item in a case where the sample is feces include fecal occult blood, Helicobacter pylori, calprotectin, Shigella, Salmonella, and enterohemorrhagic Escherichia coli. Salmonella can include at least one of Paratyphi A or Salmonella typhi. Enterohemorrhagic Escherichia coli can include at least one of O157, O111, or O26.

[Configuration Example of Diagnosis Result Screen]

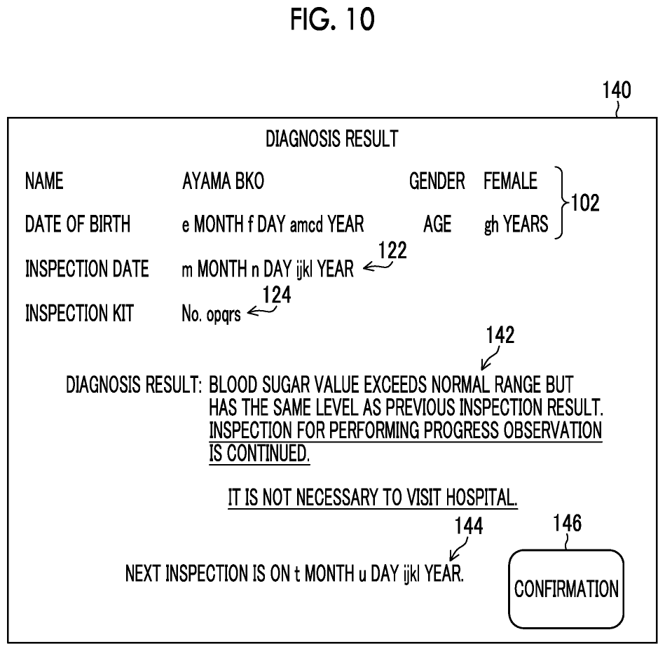

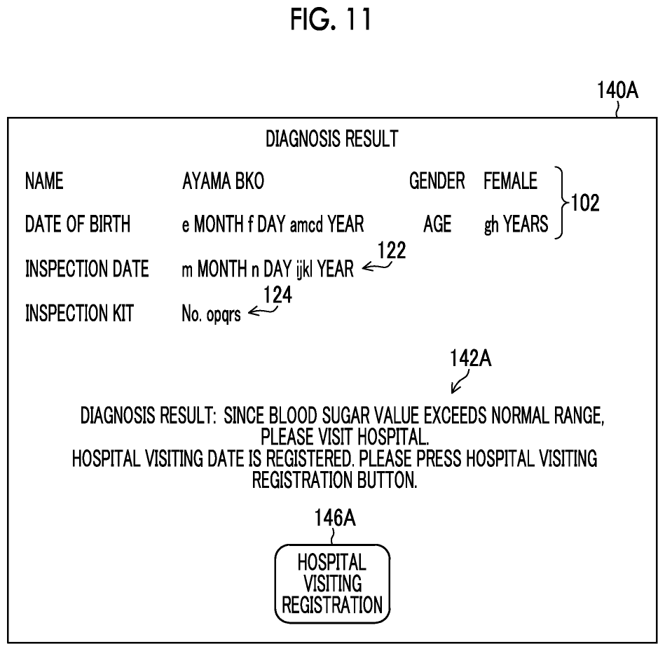

FIG. 10 is an explanatory diagram showing an example of a diagnosis result screen. FIG. 11 is an explanatory diagram showing another example of the diagnosis result screen. A diagnosis result screen 140 shown in FIG. 10 and a diagnosis result screen 140A shown in FIG. 11 are generated based on the diagnosis result transmitted from the hospital management system 14. The diagnosis result screen 140 and the diagnosis result screen 140A can be displayed by using the display unit included in the medical examinee terminal device 18 after diagnosis result reception step S136 shown in FIG. 6.

The diagnosis result screen 140 shown in FIG. 10 includes the applicant information 102, the inspection date information 122, the inspection kit information 124, diagnosis result information 142, next inspection information 144, and a confirmation button 146. Character information representing the diagnosis result is applied to the diagnosis result information 142.

The diagnosis result information 142 includes determination information of the inspection continuation. Character information indicating the inspection continuation for progress observation and the visit to the hospital are unnecessary is applied to the determination information for continuing the inspection shown in FIG. 10. The diagnosis result information 142 may describe the inspection item value used for diagnosis and the reason for diagnosis.

Character information indicating the next inspection date is applied to the next inspection information 144. In a case where the medical examinee operates the confirmation button 146, continuation confirmation information indicating that the medical examinee agrees to continue the inspection based on the content of the diagnosis result is transmitted from the medical examinee terminal device 18 to the hospital management system 14. The transmission of the continuation confirmation information corresponds to continuation confirmation information transmission step S138 shown in FIG. 6.

The diagnosis result screen 140A shown in FIG. 11 is displayed by using the display unit included in the medical examinee terminal device 18 in a case where the medical examinee is requested to visit the hospital. The diagnosis result screen 140A includes the applicant information 102, diagnosis result information 142A, and a hospital visiting registration button 146A.

The diagnosis result information 142A includes character information indicating the diagnosis result and determination information of the inspection continuation. Character information indicating that the medical examinee is encouraged to visit the hospital is applied to the determination information of the inspection continuation shown in FIG. 11. The diagnosis result information 142A may also describe the reason why it is determined that the visit to the hospital is necessary.

The hospital visiting registration button 146A is operated by the medical examinee in a case where the medical examinee registers the visit to the hospital. In a case where the hospital visiting registration button 146A is clicked, the screen is switched to the hospital visiting registration screen on which the medical examinee registers the visit to the hospital.

[Configuration Example of Hospital Visiting Registration Screen]

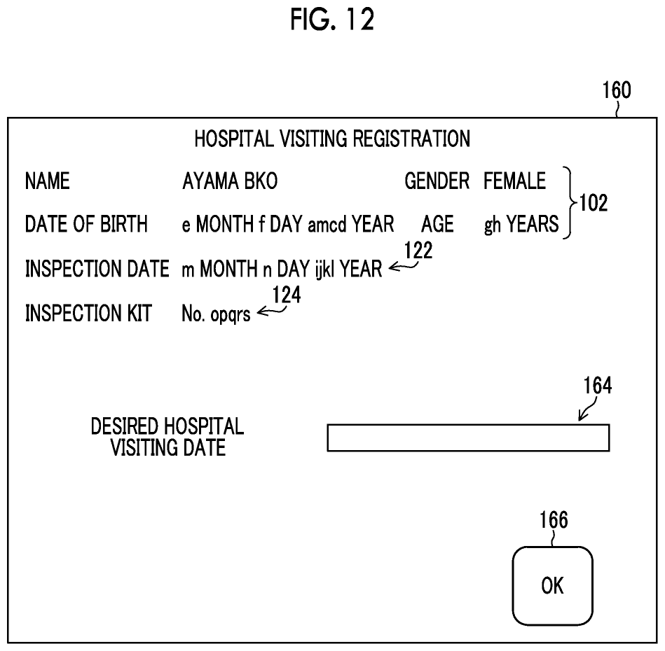

FIG. 12 is an explanatory diagram showing an example of a hospital visiting registration screen. The hospital visiting registration screen 160 shown in FIG. 12 can be displayed by using the display unit included in the medical examinee terminal device 18 based on the hospital visiting confirmation information transmitted from the hospital management system 14 to the medical examinee terminal device 18.

The applicant information 102, the inspection date information 122, the inspection kit information 124, a desired hospital visiting date input field 164, and an OK button 166 are displayed on the hospital visiting registration screen 160.

The desired hospital visiting date input field 164 is an input field in which the medical examinee inputs a desired visit to the hospital. Character input can be applied to the desired hospital visiting date input field 164. A calendar may be displayed in a case where this field is clicked and a format for selecting a date on the calendar may be applied to the desired hospital visiting date input field 164. The desired hospital visiting date input field 164 may be configured such that a desired visit time to the hospital can be input.

The OK button 166 is a button operated in a case where the medical examinee confirms the hospital visiting registration information. In a case where the OK button 166 is clicked, the hospital visiting registration information is confirmed. In a case where the OK button 166 is clicked, the hospital visiting registration information is transmitted from the medical examinee terminal device 18 to the hospital management system 14.

[Configuration Example of Hospital Visiting Reservation Screen]

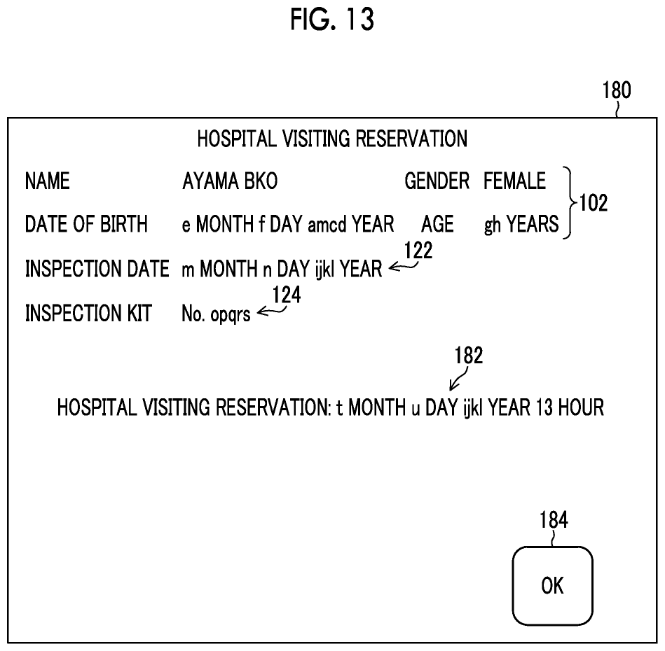

FIG. 13 is an explanatory diagram showing an example of a hospital visiting reservation screen. In a case where the hospital visiting registration information is input and the hospital visiting reservation is confirmed on the hospital visiting registration screen 160 shown in FIG. 12, a hospital visiting reservation screen 180 shown in FIG. 13 can be displayed by using the display unit included in the medical examinee terminal device 18.

The hospital visiting reservation screen 180 includes the applicant information 102, the inspection date information 122, the inspection kit information 124, hospital visiting reservation information 182, and an OK button 184. The hospital visiting reservation information 182 is character information indicating a date and time of the hospital visiting reservation registered in the hospital management system 14.

The OK button 184 is a button operated in a case where the medical examinee closes the hospital visiting reservation screen 180. In a case where the OK button 184 is clicked, the hospital visiting reservation screen 180 is closed. In a case where the OK button 184 is clicked, information indicating that the reservation is approved may be transmitted from the medical examinee terminal device 18 to the hospital management system 14.

[Configuration Example of Inspection End Screen]

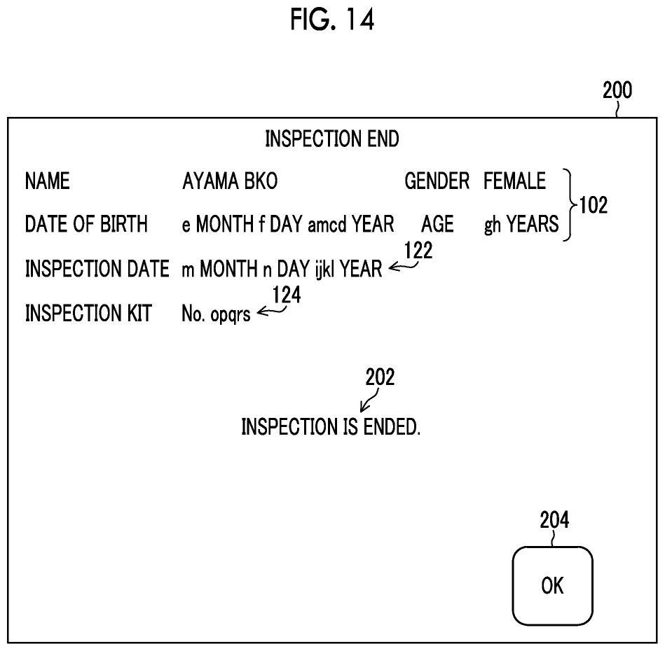

FIG. 14 is an explanatory diagram showing an example of an inspection end screen. An inspection end screen 200 shown in FIG. 14 can be displayed by using the display unit included in the medical examinee terminal device 18 in a case where the determination result is No in hospital visiting confirmation step S144 shown in FIG. 6. In hospital visiting confirmation step S144, the determination result is No in a case where the doctor determines that the subsequent inspection is not necessary.

The inspection end screen 200 includes the applicant information 102, the inspection date information 122, the inspection kit information 124, sample inspection end information 202, and an OK button 204. The sample inspection end information 202 is character information indicating that the sample inspection plan is ended.

The OK button 204 is a button operated in a case where the medical examinee closes the inspection end screen 200. In a case where the OK button 204 is clicked, the inspection end screen 200 is closed.

[Operations and Effects of Screen Display of Medical Examinee Terminal Device]

According to the screen display of the medical examinee terminal device having the aforementioned configuration, the following operations and effects can be obtained.

[1]

According to the inspection plan screen 100 shown in FIG. 8, the medical examinee can grasp the contents of the sample inspection plan that is created by the doctor and is managed by using the server device 12 from the inspection plan screen 100 displayed on the display unit of the medical examinee terminal device 18 at home of the medical examinee without going to the hospital.

[2]

According to the inspection result screen 120 shown in FIG. 9, the medical examinee can grasp the sample inspection result from the inspection result screen 120 displayed on the display unit of the medical examinee terminal device 18 at home of the medical examinee without going to the hospital and the inspection institution.

[3]

According to the diagnosis result screen 140 shown in FIG. 10 and the diagnosis result screen 140A shown in FIG. 11, the medical examinee can grasp the diagnosis result created by the doctor from the inspection result screen 120 displayed on the display unit of the medical examinee terminal device 18 at home of the medical examinee without going to the hospital and the inspection institution. According to the diagnosis result screen 140A shown in FIG. 11, the medical examinee can grasp a request of the doctor to visit the hospital.

[4]

According to the hospital visiting registration screen 160 shown in FIG. 12, in a case where the medical examinee is instructed to visit the hospital from the doctor before the next sample inspection is implemented, the desired hospital visiting date can be input by using the medical examinee terminal device 18. The medical examinee terminal device 18 can transmit information indicating the desired hospital visiting date of the medical examinee to the hospital management system 14. The hospital management system 14 can receive the information indicating the desired hospital visiting date of the medical examinee, and can confirm the hospital visiting reservation.

[5]

According to the hospital visiting reservation screen 180 shown in FIG. 13, the medical examinee can grasp the contents of the hospital visiting reservation.

[6]

According to the inspection end screen 200 shown in FIG. 14, the medical examinee can transmit information indicating that the sample inspection plan is ended from the medical examinee terminal device 18 to the hospital management system 14.

[Specific Example of Sample Inspection Kit]

Specific examples of the blood inspection kit include, for example, a biometric sample separation device described in the specification of JP3597827B and a sample analysis tool described in the specification of JP3643011B. The blood inspection kit may further include treatment tools such as a lancet, a bandage, and a disinfectant cloth, and an instruction manual used in a case where the blood is collected.

[Operations and Effects of Blood Collection Using Blood Inspection Kit]

According to the blood collection using the blood inspection kit described above, the following operations and effects can be obtained.

[1]

The blood can be collected at homes other than medical institutions.

[2]

Since the blood inspection kit is delivered on the inspection date prescribed in the sample inspection plan, forgetting to collect blood and mistakes in the blood collection date may be suppressed.

[3]

The amount of collected blood is as small as 0.1 ml or less.

[Application Example of Sample Inspection Plan Management to Animal Other than Human]

[Description of Problems]

The number of pet owners who raise pets is increasing year by year. The owner considers the pet as a member of the family, and the number of owners who care for the pets in the same manner as the human in the health management of the pets is increased. For example, in a case where a condition of the pet is different from usual and there is a concern that the pet is abnormal, it has become commonplace that the owner immediately takes the pet to a veterinary hospital and the pet receives the medical inspection by a veterinarian.

However, the owner may not notice the abnormal condition of the pet, and even though the medical condition of the pet is worse, the medical condition of the pet may be continuously worse. That is, although the owner pays attention to the health management of the pet like the human, a system that manages the inspection plan created by the veterinarian has not been established.

[Configuration Example of Sample Inspection Management System for Animal Other than Human]

The sample inspection plan management system 10 and the sample inspection plan management system 10A described with reference to FIGS. 1 to 14 may be applied to the animal other than the human by replacing the doctor with the veterinarian, the hospital with the veterinary hospital, and the medical examinee with the owner. The veterinarian is an example of the doctor.

The animal other than the human may include animals raised in zoos, animals such as chickens, pigs, cows, and horses raised by livestock farmers such as ranches, and racehorses. The owner may include a person who manages the health of the animal such as a caretaker or a livestock manager.

Examples of the animal other than the human include mammals such as dogs, cats, pigs, horses, cows, hamsters, guinea pigs, rabbits, squirrels and ferrets, birds, and reptiles such as snakes and lizards. Examples of a sample of the animal other than the human include blood, urine, feces, saliva, mucous membranes, and body hair. Examples of a sample inspection item in a case where the sample is urine include urine pH, urinary protein, urine occult blood, urine specific gravity, urine sugar, acesmin, aldehydes, ketone bodies, and creatinine.

Although the sample inspection plan management system has been described in the present embodiment, the sample inspection plan management device, the sample inspection plan management method, and the program can be used similarly to the sample inspection plan management system for the human described above.

[Operations and Effects of Sample Inspection Management System for Animal Other than Human]

According to the sample inspection management system having the aforementioned configuration, the following operations and effects can be obtained.

[1]

It is possible to manage a sample inspection plan created by a veterinarian in the same manner as the sample inspection plan management system for the humans. Accordingly, it is possible to implement a regular sample inspection of the pet without taking the pet to the veterinary hospital.

[2]

Even though the owner does not notice the abnormal condition of the pet, it is possible to restrain the medical condition of the pet from being worse and being continuously worse.

[3]

The owner can grasp the contents of the sample inspection plan and the veterinary diagnosis result by looking at the screen displayed on the display unit of the owner terminal device.

The application example to the animal other than the human shown in the present embodiment can be applied to a regular health diagnosis of the pet. For example, the aforementioned embodiment can be applied to a case where the medical inspection is implemented by the veterinarian for some kinds of illness, and medication and treatment during the medical inspection are received. The aforementioned embodiments can be applied to a case where the veterinarian determines that regular progress observation is necessary for six months or one year by continuing to take medicines and supplements that suppress diseases that are likely to develop due to aging in advance.

[Application Example to Program Causing Computer to Function as Sample Inspection Plan Management Device]

The above-described sample inspection plan management device and method can be realized by executing a program causing a computer to realize a function corresponding to each unit in the sample inspection plan management device and a function corresponding to each step in the sample inspection plan management method. For example, a sample inspection plan management program causing a computer to realize a sample inspection plan acquisition function, a delivery information output function, and an inspection result acquisition function can be realized.

It is possible to store the program causing the computer to realize the above-described sample inspection plan management function in a computer-readable information storage medium which is a tangible non-transitory information storage medium, and it is possible to provide the program through the information storage medium.

Instead of an aspect of storing the program in the non-transitory information storage medium and providing the program, an aspect of providing a program signal via a network can also be realized.

### Combinations of Embodiments and Modification Examples

The components described in the above-described embodiment and the components described in the application examples can be appropriately used in combination, and some of the components can be replaced.

In the above-described embodiment of the present invention, components can be appropriately changed, added, or deleted without departing from the spirit of the present invention. The present invention is not limited to the above-described embodiments, and can be variously modified by those having ordinary knowledge in the art within the technical spirit of the present invention.

### EXPLANATION OF REFERENCES

10: sample inspection plan management system
10A: sample inspection plan management system
12: server device
14: hospital management system
16: hospital terminal device
18: medical examinee terminal device
20: inspection institution terminal device
22: database
24: network
30: information acquisition unit
30A: inspection plan acquisition unit
30B: inspection result acquisition unit
32: information output unit
32A: delivery information output unit
32B: inspection result output unit
34: scheduling unit
36: analysis unit
40: controller
42: memory
44: storage device
46: network controller
48: power supply device
49: bus
50: integrated platform
60: business system
62: electronic medical record system
64: ordering system
70: medical inspection system
72: radiation department system
74: sample inspection department system
100: inspection plan screen
102: applicant information
104: sample kind information
106: inspection number-of-times information
108: inspection frequency information
110: inspection start date information
112: inspection kit delivery destination information
114: inspection result delivery necessity information
116: OK button
120: inspection result screen
122: inspection date information
124: inspection kit information
126: inspection result list
140: diagnosis result screen
140A: diagnosis result screen
142: diagnosis result information
142A: diagnosis result information
144: next inspection information
146: inspection continuation confirmation button
146A: hospital visiting registration button
160: hospital visiting registration screen
164: desired hospital visiting date input field
166: OK button
180: hospital visiting reservation screen
182: hospital visiting reservation information
184: OK button
200: inspection end screen
202: sample inspection end information
204: OK button
S10 to S148: each step of sample inspection management method

What is claimed is:

1. A sample inspection plan management device that manages a sample inspection plan in a case where a sample inspection is implemented by using a sample inspection kit acquirable by a medical examinee outside a hospital, the device comprising:
an inspection plan acquisition unit that acquires a sample inspection plan created by a doctor via a system of the hospital;
a delivery information output unit that outputs delivery information including a delivery destination of the sample inspection kit based on the sample inspection plan; and
an inspection result acquisition unit that acquires an inspection result of a sample collected by using the sample inspection kit delivered based on the delivery information via a system of an inspection institution that implements the sample inspection by retrieving the sample inspection kit.

2. The sample inspection plan management device according to claim 1, further comprising:
an inspection result output unit that outputs the inspection result acquired by using the inspection result acquisition unit to the system of the hospital.

3. The sample inspection plan management device according to claim 2,
wherein the inspection result output unit outputs the inspection result acquired by using the inspection result acquisition unit to a medical examinee terminal device used by the medical examinee.

4. The sample inspection plan management device according to claim 1,
wherein the inspection plan acquisition unit acquires the sample inspection plan including at least one of a kind of the sample, a frequency of the sample inspection, or the number of times of the sample inspection.

5. The sample inspection plan management device according to claim 1,
wherein the inspection plan acquisition unit acquires the sample inspection plan created by the doctor by using, as the sample, at least one of blood of human, feces of human, urine of human, blood of animal other than human, feces of animal other than human, or urine of animal other than human.

6. The sample inspection plan management device according to claim 1,
wherein the delivery information output unit outputs the delivery information to a sample inspection kit providing institution that provides the sample inspection kit.

7. The sample inspection plan management device according to claim 1,
wherein the delivery information output unit outputs the delivery information to the inspection institution.

8. The sample inspection plan management device according to claim 1,
wherein the delivery information output unit outputs the delivery information having, as the delivery destination, any of home of the medical examinee, a pharmacy, a store other than the pharmacy, a business office of a delivery company, and a post office.

9. A sample inspection plan management system comprising a server device connected to communicate with a network,
wherein the server device is a sample inspection plan management device that manages a sample inspection plan in a case where a sample inspection is implemented by using a sample inspection kit acquirable by a medical examinee outside a hospital,
the server device including
an inspection plan acquisition unit that acquires a sample inspection plan created by a doctor via a system of the hospital,
a delivery information output unit that outputs delivery information including a delivery destination of the sample inspection kit based on the sample inspection plan, and
an inspection result acquisition unit that acquires an inspection result of a sample collected by using the sample inspection kit delivered based on the delivery information via a system of an inspection institution that implements the sample inspection by retrieving the sample inspection kit.

10. The sample inspection plan management system according to claim 9, further comprising:
a medical examinee terminal device that is connected to communicate with the network, and is used by the medical examinee,
wherein the medical examinee terminal device includes a display unit that displays at least one of a kind of the sample applied to the sample inspection, a frequency of the sample inspection, or the number of times of the sample inspection.

11. The sample inspection plan management system according to claim 10,
wherein the display unit displays a result of the sample inspection.

12. The sample inspection plan management system according to claim 9, further comprising:
a providing institution terminal device that is connected to communicate with the network, and is provided in a sample inspection kit providing institution that provides the sample inspection kit,
wherein the delivery information output unit outputs the delivery information to the providing institution terminal device.

13. The sample inspection plan management system according to claim 9, further comprising:
an inspection institution terminal device that is connected to communicate with the network, and is provided in the inspection institution,
wherein the delivery information output unit outputs the delivery information to the inspection institution terminal device.

14. A sample inspection plan management method of managing a sample inspection plan in a case where a sample inspection is implemented by using a sample inspection kit acquirable by a medical examinee outside a hospital, the method comprising:
an inspection plan acquisition step of acquiring a sample inspection plan created by a doctor via a system of the hospital;
a delivery information output step of outputting delivery information including a delivery destination of the sample inspection kit based on the sample inspection plan; and
an inspection result acquisition step of acquiring an inspection result of a sample collected by using the sample inspection kit delivered based on the delivery information via a system of an inspection institution that implements the sample inspection by retrieving the sample inspection kit.

15. A non-transitory computer readable recording medium causing a computer to realize a sample inspection plan management function of managing a sample inspection plan in a case where a sample inspection is implemented by using a sample inspection kit acquirable by a medical examinee outside a hospital in a case where a command stored in the recording medium is read by a computer, the sample inspection plan management function comprising:
an inspection plan acquisition function of acquiring a sample inspection plan created by a doctor via a system of the hospital;
a delivery information output function of outputting delivery information including a delivery destination of the sample inspection kit based on the sample inspection plan; and
an inspection result acquisition function of acquiring an inspection result of a sample collected by using the sample inspection kit delivered based on the delivery information via a system of an inspection institution that implements the sample inspection by retrieving the sample inspection kit.
